(19) United States
(12) Patent Application Publication
(10) Pub. No.: US 2008/0251598 A1
(43) Pub. Date: Oct. 16, 2008

(54) Wick Assemblies

(76) Inventor: Peter William Ross, Crowthorne (GB)

Correspondence Address: STITES & HARBISON PLLC, 1199 NORTH FAIRFAX STREET, SUITE 900, ALEXANDRIA, VA, 22314, US

(21) Appl. No.: 12/066,864

(22) PCT Filed: Sep. 14, 2006
(86) PCT No.: PCT/GB06/03396

§ 371 Date: Jun. 4, 2008

(30) Foreign Application Priority Data
Sep. 15, 2005 (GB) 0518866.9

(51) Int. Cl. A61L 9/02

(52) U.S. Cl. 239/44

(57) ABSTRACT

A wick assembly for, for example, a fragrance dispenser such as an air freshener comprises a conductive wick (8) that wicks the liquid from a source (1). The liquid is evaporated from the wick (8) when connected to a power source to heat the wick.

The invention relates to wick assemblies and, in particular, wick assemblies for use in vapour dispensers such as air fresheners, insecticide dispensers and personal fragrance dispensers.

A known wick assembly for a vapour dispenser such as an air freshener comprises a wick for conveying a liquid from a source and a heated ceramic collar surrounding, but not in contact with, a portion of the wick remote from the liquid. The ceramic collar is connected to a source of power that heats the collar so heating the wick in turn by convection to evaporate liquid from the wick. It is a disadvantage of such an arrangement that the ceramic collar has significant thermal mass. In addition, the gap between the collar and the wick slows the transfer of heat from the collar to the wick. Accordingly, when power is supplied to the collar, there is a time delay before the liquid is evaporated. This can be disadvantageous since, for example, it is not apparent whether the device is working or not when it is first switched on. In addition, evaporation takes place only from that portion of the surface of the wick that is heated by the collar and this provides only a limited area for evaporation.

According to the invention, there is provided a wick assembly for dispersing a liquid comprising a heatable wick for absorbing a liquid from a source and a power supply for heating the wick intermittently to disperse liquid from the heatable wick, the heatable wick comprising conductive fibres and the power supply heating the conductive fibres of the heatable wick such that during heating, all or most of the liquid in the wick is dispersed and when not being heated, the heatable wick absorbs liquid from the source.

The following is a more detailed description of some embodiments of the invention, by way of example, reference being made to the accompanying drawings in which:

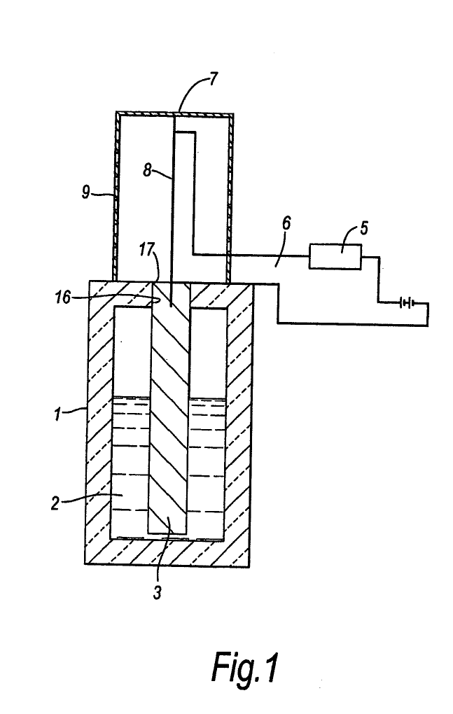

FIG. 1 is a side elevation, partly in section, of an air freshener showing a fragrance container with a thin wick incorporating a resistive heater, a control circuit and battery power supply

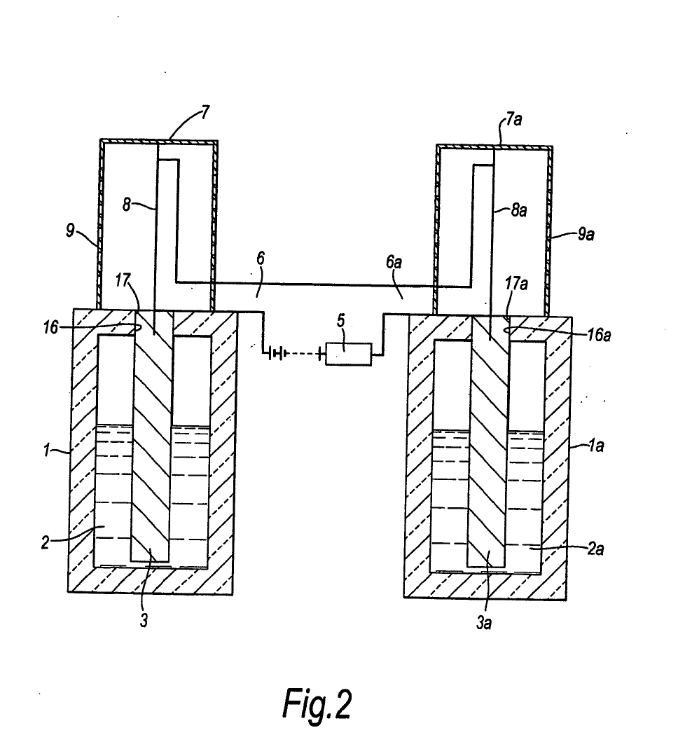

FIG. 2 is a schematic view with an alternative form of the air freshener of FIG. 1 with two fragrance containers

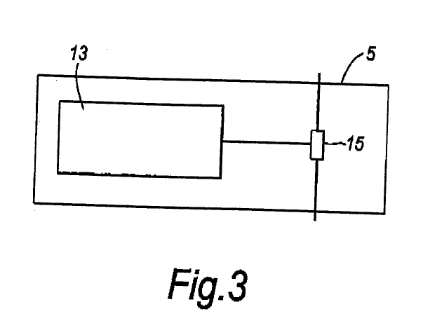

FIG. 3 is a diagram of a section of an air freshener as shown if FIGS. 1 and 2, showing an arrangement for connecting the heated wick to the capillary wick.

FIG. 4 is a diagram of a drive circuit for any of the forms of air freshener shown in FIGS. 1 and 2.

Referring first to FIGS. 1, the air freshener includes a fragrance container 1. The container 1 is formed from any suitable material such as glass or plastics and has a fragrance-containing chamber leading to a neck 16 terminating in an open end 17. An internal capillary wick 3, which may be formed from any suitable known wicking material, is held in the neck 16 and has a first end and a second end. The wick 3 depends downwardly so that the first end is in the fragrance 2, which may be of any known kind. The wick 3 may have a diameter of from 3 mm to 13 mm. The second end extends through the neck 16 and terminates at the open end 17. The wick 3 may terminate substantially flush with the open end 17 of the neck 16 of the container 1 so that the container 1 may be provided with a simple tear-off closure (not shown) covering the open end 17 to prevent loss of fragrance during transport and sale of the air freshener. The closure may be attached to the container 1 by adhesive or welding.

Since there is no space between a foil seal and the wick, there is no possibility of fragrance accumulating in this area so that there is no spillage of fragrance 2 when the foil is removed, prior to insertion into the housing 9.

The air freshener also includes a housing 9, which may be formed from any suitable material, such as a plastics material. The housing is of generally cylindrical shape with an open end and a closed end. The housing 9 is mountable on the container 1 so that the open end covers the open end 17 (after removal of any closure) of the container 1 and the housing 9 is generally co-axial with the axis of the neck 16. The housing 9 is provided with holes or slots for the easy flow of air into and out of the housing 9. The housing 9 has a mounting 7 at the closed end and the mounting 7 carries a first end of a heatable wick 8. The heatable wick 3 extends axially along the housing 9 and has a second end extending into the capillary wick 3 in a manner to be described below. The heatable wick 8 is formed from one or more strands of non-conductive fibres some or all of which have been treated to be electrically conductive. The strands are, for example, twisted or plaited together. Such fibres can be obtained for example from Bekaert Advanced Materials and have a resistance when twisted or plaited in the final assembly between 0.2 and 5000 ohms.

A power supply comprises a number of dry-cell batteries 4 connected in series to a pulse circuit 5 that is, in turn, connected across the heatable wick 8 by wires 16. The pulse circuit 5 will be described in more detail below. A heatable wick 8 with a resistance in the range 2 to 100 ohms more preferably 20 to 60 ohms is particularly suitable for use with battery power supplies.

In use, the heatable wick 8 absorbs fragrance fluid from the capillary wick 3. The fragrance fluid flows along the heatable wick 8. When power is supplied from batteries 4 through the pulse circuit 5 via connection wires 6 to the heatable wick 8, the conductive fibres in the heatable wick 8 heat up to vaporise the fragrance liquid absorbed by the heatable wick 8. Since the heat is applied at the wick 8, the heat reaches the fragrance liquid very quickly by conduction and is applied over a large area of the fragrance. The liquid may be heated fast enough to generate a small cloud of fragrance liquid droplets that is generally visible to the user. After a heating pulse from the pulse circuit 5, there is a no pulse period and so the heatable wick 8 is allowed to cool, and fragrance fluid 2 is drawn from the capillary wick 3 to the heatable wick 8 to be vaporised by the next successive pulse from the circuit 5. Again, the area of the conductive fibres is such that the heat disperses quickly. The length of the heating pulses provided by the pulse circuit 5 depends on the dimensions and resistance of the heatable wick 8 but is in the range 0.1 seconds to 20 seconds more preferably 1 to 10 seconds. In this mode, therefore, the heatable wick 8 and the power supply may be chosen so that the length and power of the pulse is just sufficient to disperse all, or substantially all, of the liquid drawn up by the heatable wick 8 from the capillary wick 3. Between each pulse and the next successive pulse, the heatable wick 8 draws-up further liquid from the capillary wick 3 which is then dispersed by the subsequent pulse. This allows the heatable wick 8 to be much thinner than the capillary wick 3 and also allows a low power source, such as batteries, to be used for the power supply. For example, the heatable wick 8 may be between 5% and 50% more preferably 10% to 40% of the thickness of the capillary wick 3.

FIG. 2 shows a dual fragrance system, allowing two different fragrances to be generated at different times. The concept for such a device is described in WO 2004096300. The system of FIG. 2 has an air freshener of the kind described with reference to FIG. 1 and a second such air freshener. The parts of the first air freshener are given the same reference numerals in FIG. 2 and in FIG. 1 and the second air freshener has those parts with the same reference numerals but with the suffix “a”. There is a single power supply 5 generally as described above with reference to FIG. 1 but with connections 6a connecting a second output of the pulse circuit 5 across the second heatable wick 8a. The power circuit 5 pulses the heatable wicks 8, 8a independently at pre-programmed spaced time intervals to avoid olfactory fatigue. To reduce cost of manufacture, there may be a single housing 9 with the heatable wicks 8, 8a formed from a continuous length of fibres with means to confine each fragrance to a respective part of the length of the fibres, such as a clamp or waxy deposit. A common electrode may contact the wick at this point to make electrical connection.

FIG. 3 shows an arrangement for making connection between the heatable wick 8, and the capillary wick 3. The heatable wick 8, needs to have good physical connection with the capillary wick 3, and this is achieved by capturing both ends of the heatable wick 8 in a clamp 10 with a narrow profile 11 that allows the heatable wick 8 to be inserted into the capillary wick 3. The clamp 10 holds the heatable wick 8, and allows flow of fragrance 2 from the capillary wick 3 to the heatable wick 8. When the fragrance 2 has all been evaporated it is a simple matter to disengage the container 1, from the housing 9, 9a and replace it with a fresh container 1, after first having removed any closure. This arrangement can also be used with the arrangement of FIG. 2 having dual fragrances.

FIG. 4 shows a simple pulse circuit 5 for providing power to the heatable wick 8. This circuit uses a pulse generator 13 to power a transistor 15 or other switching device. The transistor 15 is driven fully on by the pulse signal and so the maximum current is supplied to the heatable wick 8. The pulse may be from 0.1 to 20 seconds long, and use a power of between 0.05 and 20 Watts. There may be means for preventing the heated wick 8 exceeding a predetermined temperature. Again, this circuit 5 can also be used with the arrangement of FIG. 2 having dual fragrances but include two transistors or other switching devices each controlling a respective heatable wick 8, 8a.

The dispensers described above with reference to the drawings can be used for other purposes such as dispersal of insecticide or other evaporable substances required in low concentrations inside buildings. Another application is for personal fragrance dispensers, where a perfume or similar substance is dispersed in a cloud of droplets from a dispenser close to the skin. The dispenser may be made in a small portable form if required, and hung from clothing, attached to the skin with a temporary adhesive or hung on a chain as an adornment if required.

1. A wick assembly for dispersing a liquid comprising a heatable wick (8) for absorbing a liquid from a source (1) and a power supply (4, 5) the heatable wick (8) being formed of fibres including at least some electrically conductive fibres and the power supply providing power intermittently to the conductive fibres of the heatable wick (8) to heat the conductive fibres such that during heating, all or most of the liquid in the heatable wick (8) is dispersed and when not being heated, the heatable wick (8) absorbs liquid from the source.

2. An assembly according to claim 1 wherein the conductive fibres are formed from one or more continuous lengths of fibre.

3. An assembly according to claim 2 wherein the conductive fibres are in the form of a narrow wick made by twisting.

4. An assembly according to claim 2 wherein the conductive fibres are in the form of a narrow wick made by plaiting.

5. An assembly according to claim 34 wherein the heatable wick (8) is between 5 mm and 300 mm in length

6. An assembly according to claim 3 wherein the resistance of the wick is between 0.2 and 5000 Ohms.

7. An assembly according to claim 6 wherein the conductive fibres are formed from non-conductive fibres coated or plated with a conductive material

8. An assembly according to claim 1 wherein said heatable wick (8) is held in a housing (9) provided with apertures for the passage of air through the housing (9).

9. An assembly according to claim 8 wherein the housing (9) includes means for supplying electrical power to the wick from the power supply (4, 5).

10. An assembly according to claim 9 wherein the power is supplied in timed pulses from a control circuit (5) of the power supply (4, 5).

11. An assembly according to claim 10 wherein the power supply (4, 5) applies power for a period between 0.1 seconds and 20 seconds

12. An assembly according to claim 1 wherein the heatable wick (8) is formed from conductive fibres and non-conductive fibres.

13. An assembly according to claim 1 wherein the heatable wick (8) is formed wholly from conductive fibres.

14. An assembly according to claim 1 in combination with at least one container (1) for a liquid.

15. As assembly according to claim 14 wherein the container (1) includes an internal capillary wick (3) and the capillary wick (3) is connected to the heatable wick (8).

16. An assembly according to claim 15 wherein the heatable wick (8) is held in a housing (9), the housing (9) being connected to the container (1).

17. An assembly according to claim 16 wherein the container (1) and the internal capillary wick (1 3) are separable from the housing (9) to allow replacement of the container (1) and the internal capillary wick (3).

18. An assembly according to claim 13 wherein two containers (1, 1a) are provided, each container connecting to a respective heatable wick (8, 8a) that is separately controlled by the power supply (4, 5).

19. An assembly according to claim 14 in which, fragrance container (1) includes a neck (16) having an open end (1 7) the internal capillary wick (3) having a top arranged to be substantially level with the open end (17) of the neck (1 6) of the fragrance container (1) and the container (1) is supplied with a tear-off closure covering said open end (17) attached to the surface by adhesive or by welding.

20. An assembly according to claim 1 including means to prevent the temperature of the or each heatable wick (8, 8a) exceeding a predetermined temperature.

21. An assembly according to claim 1 in which the power supply includes a primary or secondary battery source (4).

22. An air freshener incorporating a wick assembly according to claim 1.

23. A vapour dispenser incorporating a wick assembly according claim 1.

24. An insecticide dispenser incorporating a wick assembly according to claim 1.

25. A personal fragrance dispenser incorporating a wick assembly according to claim 1.
